(19) United States
(12) Patent Application Publication
(10) Pub. No.: US 2015/0018631 A1
(43) Pub. Date: Jan. 15, 2015

(54) Apparatus and Method for Measuring Physiological Signals

(71) Applicant: AVITA CORPORATION, New Taipei City (TW)

(72) Inventor: Chun-Ho Lee, New Taipei City (TW)

(21) Appl. No.: 13/941,539

(22) Filed: Jul. 14, 2013

(51) Int. Cl. A61B 5/00; A61B 5/0402; A61B 5/024

(52) U.S. Cl. CPC A61B 5/7282; A61B 5/024; A61B 5/0402; A61B 5/7235
USPC 600/301; 600/500

(57) ABSTRACT

A measuring unit has at least one first signal-measuring end and at least one second signal-measuring end. The first signal-measuring end and the second signal-measuring end contact at least two symmetrical portions of a living being to obtain at least one first pulse signal and at least one second pulse signal of the two symmetrical portions, respectively. A signal-analyzing unit is coupled to the measuring unit. The signal-analyzing unit obtains at least one physiological data based on the first pulse signal and the second pulse signal, respectively, further to determine a physiological condition of the living being according to the physiological data.

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### BACKGROUND OF THE PRESENT INVENTION

1. Field of Invention

This invention relates to an apparatus for measuring a physiological signal and, more particularly, to an apparatus and method for measuring a physiological signal capable of accurately assessing a physiological condition by simultaneously contacting at least two symmetrical portions of a living being.

2. Description of the Related Art

Arteriosclerosis is a general term for a condition characterized by thickening, hardening, loss of elasticity of arterial walls, narrowing of vessel lumens, or hyperplasia. Age growth is the main risk factor of the arteriosclerosis.

Usually the arteriosclerosis may take place unconsciously. Any symptom may not be generated before angiemphraxis reaches 50%. However, once the angiemphraxis exceeds 75%, angina pectoris or other symptoms may be generated, thus posing a threat to life. Accordingly, it is important for prevention of cardiovascular disease to know the degree of arteriosclerosis of oneself.

Currently a pulse wave velocity (PWV) is considered as one standard method for assessing the degree of arteriosclerosis using a non-invasive method in the medical field. Further, research reports point out that the pulse wave velocity is an important index of preventing heart disease, apoplexy, and cardiovascular disease.

FIG. 1 is a schematic diagram of pulse signals of two asymmetrical portions (such as a finger and a toe) at a single side in a prior art. In FIG. 1, a photoplethysmography (PPG) may be used for simultaneously measuring pulse signals of two asymmetrical portions (such as a finger and a toe) at a single side, thus to obtain a pulse wave velocity of the two asymmetrical portions (such as the finger and the toe) at the single side based on a pulse transit time (ΔT) of the two asymmetrical portions (such as the finger and the toe) at the single side to indicate the degree of arteriosclerosis using the following formula (1).

$$PWV = \frac{( {L}_{1} + {L}_{2} )}{Δ T} \qquad ( 1 )$$

FIG. 2 is a schematic diagram of pulse signals in different degrees of arteriosclerosis of two asymmetrical portions (such as a finger and a toe) at a single side in a prior art. In FIG. 2, when the arteriosclerosis of the finger of a tested body is more serious than that of the toe, the pulse signal of the finger is transmitted faster than that of the toe. Accordingly, a pulse transit time between the pulse signals of the finger and the toe may increase (from Δt to Δt′) thus to decrease a pulse wave velocity, and therefore determination may be inaccurate.

FIG. 3 is a schematic diagram of a pulse signal of a single portion (such as a finger) at a single side in a prior art. In FIG. 3, a photoplethysmography may be used for measuring the pulse signal of the single portion (such as the finger). Characteristic points (such as a first peak and a second peak) of a systolic wave and a diastolic wave of the pulse signal are obtained, thus to assess arteriosclerosis. In detail, the peak height (b) of the diastolic wave is divided by the peak height (a) of the systolic wave using the formula (2) to obtain a reflection index (RI), and the body height (m) of a tested body is divided by a time difference (T_{DVP}) between the peak of the diastolic wave and the peak of the systolic wave using the formula (3) to obtain a stiffness index (SI). The degree of arteriosclerosis can be determined based on the reflection index and the stiffness index.

$$\begin{matrix}RI = \frac{b}{a} \times 100 \% & ( 2 ) \\ SI = \frac{bodyheight}{{T}_{DVP}} ( m \text{/} sec ) & ( 3 )\end{matrix}$$

Compared with the calculation of a pulse wave velocity, the calculations of the reflection index and the stiffness index are more convenient and can avoid errors caused by measuring artery distances since they can be obtained just based on the pulse signal of a single portion (such as a finger) at a single side. Further, the reflection index and the stiffness index have been considered as effective reference index of assessing arteriosclerosis clinically.

FIG. 4 is a schematic diagram of pulse signals of a single portion (such as a finger) at a single side of four tested bodies having different ages and diseases, respectively, in a prior art. In FIG. 4, a characteristic point (such as a second peak) of a diastolic wave becomes unobvious due to age growth and diseases. Either the age growth (Class B and Class C) or the cardiovascular disease (Class D) may make the characteristic point (such as the second peak) of the diastolic wave more and more unobvious, and therefore neither a first-order differential method nor a second-order differential method fails to accurately locate the characteristic point (such as the second peak) of the diastolic wave. Accordingly, assessment based on the reflection index and the stiffness index is only effective for the healthy young person (Class A) and the healthy middle-aged person (Class B).

Although assessment of the arteriosclerosis based on the pulse wave velocity or the reflection index and the stiffness index has a good clinical performance and is published by international medical journals as well, it should be still improved.

First, when pulse signals of two asymmetrical portions (such as a finger and a toe) at a single side are simultaneously measured via a photoplethysmography, if the hardening of one portion is more serious than that of the other portion, the pulse transit time between the pulse signals of the finger and the toe may be inaccurate, thus affecting the accuracy of the pulse wave velocity.

Second, when a pulse signal of a single portion (such as a finger) at a single side is measured via a photoplethysmography, if a pulse signal at the other side of a tested body is to be measured, the photoplethysmography has to be reset, thus increasing the measuring time and the operation complexity.

Third, only a certain number of pulse wave velocities are used to assess the degree of arteriosclerosis. However, physiological variation is dynamic and complex. Accordingly, it is a development tendency to quantify the complexity of arteriosclerosis in a dynamic view.

Fourth, a characteristic point (such as a peak) of a diastolic wave of a single portion (such as a finger) at a single side may gradually become smooth due to age growth and diseases, thus affecting the accuracy of the reflection index and the stiffness index.

This invention is to improve the prior art.

### SUMMARY OF THE PRESENT INVENTION

The invention provides an apparatus and method for measuring a physiological signal to improve accuracy of assessing a physiological condition.

According to one aspect of the invention, the invention provides an apparatus for measuring a physiological signal including a measuring unit and a signal-analyzing unit. The measuring unit has at least one first signal-measuring end and at least one second signal-measuring end. The first signal-measuring end and the second signal-measuring end contact at least two symmetrical portions of a living being to obtain at least one first pulse signal and at least one second pulse signal of the two symmetrical portions, respectively. The signal-analyzing unit is coupled to the measuring unit. The signal-analyzing unit obtains at least one physiological data based on the first pulse signal and the second pulse signal, respectively, further to determine a physiological condition of the living being according to the physiological data.

According to another aspect of the invention, the invention provides a method for measuring a physiological signal. The method includes the following steps: contacting at least two symmetrical portions of a living being to obtain at least one first pulse signal and at least one second pulse signal of the two symmetrical portions, respectively; obtaining at least one physiological data based on the first pulse signal and the second pulse signal, respectively, further to determine a physiological condition of the living being according to the physiological data.

These and other features, aspects, and advantages of the present invention will become better understood with regard to the following description, appended claims, and accompanying drawings.

### BRIEF DESCRIPTION OF THE DRAWINGS

FIG. 1 is a schematic diagram of pulse signals of two asymmetrical portions (such as a finger and a toe) at a single side in a prior art;

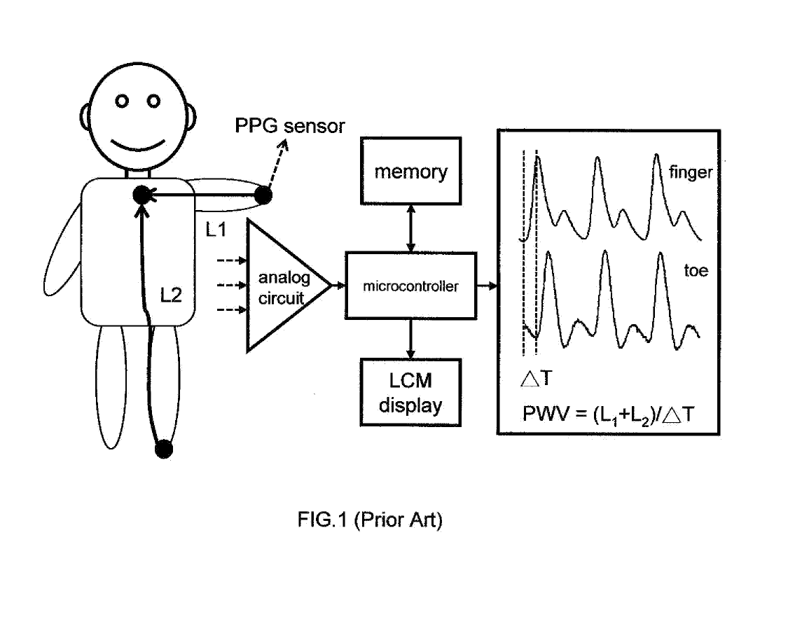

FIG. 2 is a schematic diagram of pulse signals in different degrees of arteriosclerosis of two asymmetrical portions (such as a finger and a toe) at a single side in a prior art;

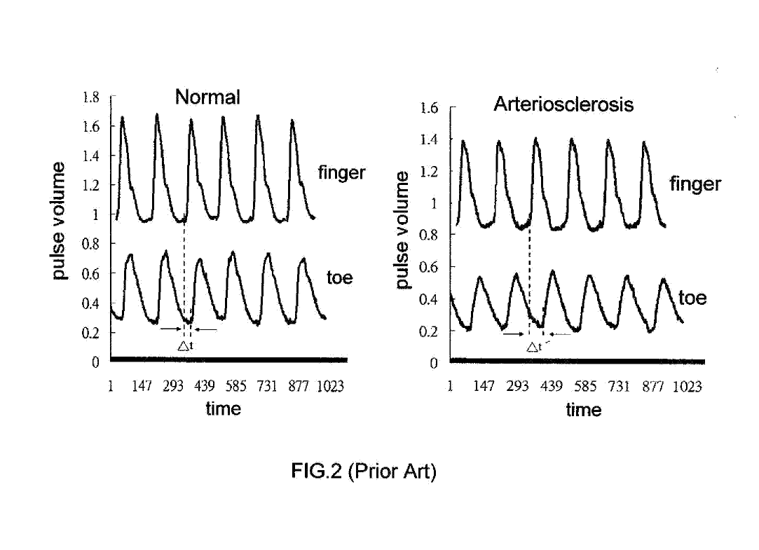

FIG. 3 is a schematic diagram of a pulse signal of a single portion (such as a finger) at a single side in a prior art;

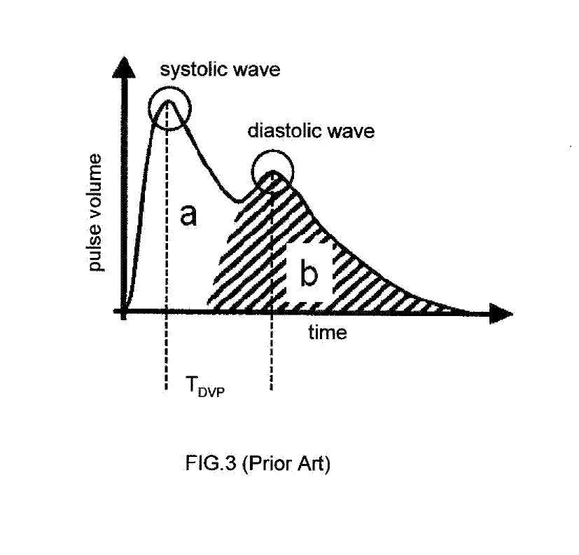

FIG. 4 is a schematic diagram of pulse signals of a single portion (such as a finger) at a single side of four tested bodies having different ages and diseases, respectively, in a prior art;

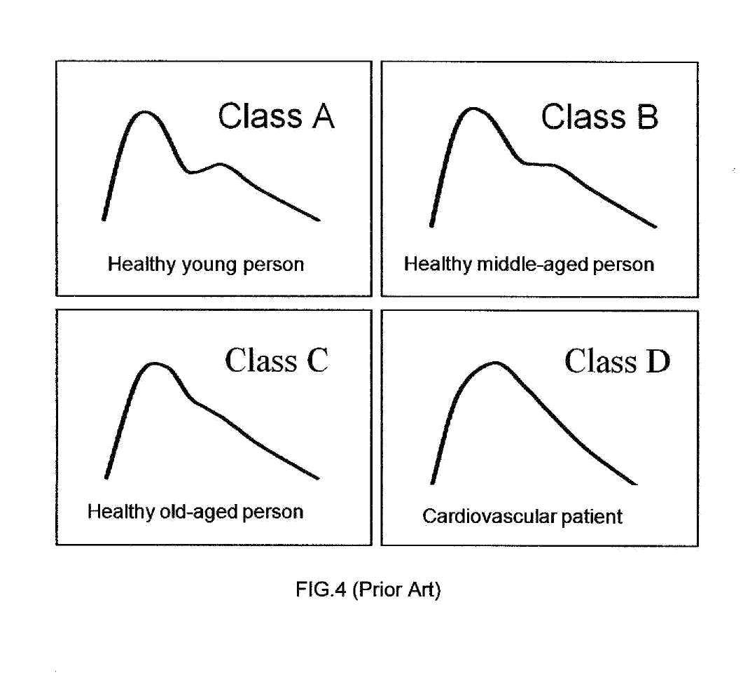

FIG. 5 is a schematic diagram of an apparatus for measuring a physiological signal according to one embodiment;

FIG. 6 is a schematic diagram of calculation of a first crest-to-cycle ratio of a first pulse signal or a second crest-to-cycle ratio of a second pulse signal according to one embodiment;

FIG. 7 is a schematic diagram of calculation of a first pulse wave velocity based on a first pulse signal along with an ECG signal and a second pulse wave velocity based on a second pulse signal along with an ECG signal according to one embodiment;

FIG. 8 is a schematic diagram of calculation of a first multiscale entropy coefficient based on a first pulse signal along with an ECG signal or a second multiscale entropy coefficient based on a second pulse signal along with an ECG signal according to one embodiment;

FIG. 9 is a schematic diagram of calculation of a coarse-grained technology according to one embodiment;

FIG. 10 is a schematic diagram of sample entropy related to scale variability according to one embodiment; and

FIG. 11 is a schematic diagram of calculation of a first pearson correlation coefficient based on a first pulse signal along with an ECG signal or a second pearson correlation coefficient based on a second pulse signal along with an ECG signal according to one embodiment.

### DETAILED DESCRIPTION OF THE PREFERRED EMBODIMENT

In the prior art, a physiological condition (such as arteriosclerosis) is assessed according to pulse signals of two asymmetrical portions (such as a finger and a toe) at a single side or according to a pulse signal of a single portion (such as a finger) at a single side. The invention provides an apparatus for measuring a physiological signal to improve the prior art. The apparatus includes a measuring unit and a signal-analyzing unit. The measuring unit has at least one first signal-measuring end and at least one second signal-measuring end. The first signal-measuring end and the second signal-measuring end contact at least two symmetrical portions of a living being to obtain at least one first pulse signal and at least one second pulse signal of the two symmetrical portions, respectively. The signal-analyzing unit is coupled to the measuring unit. The signal-analyzing unit obtains at least one physiological data based on the first pulse signal and the second pulse signal, respectively, further to determine a physiological condition of the living being according to the physiological data. The embodiments of the invention are not limited to one kind of symmetrical portions (such as ears), and the embodiments can also be adapted to different kinds of symmetrical portions (such as ears and fingers, or ears, fingers, and toes). In the following embodiments, the ears may be taken for example.

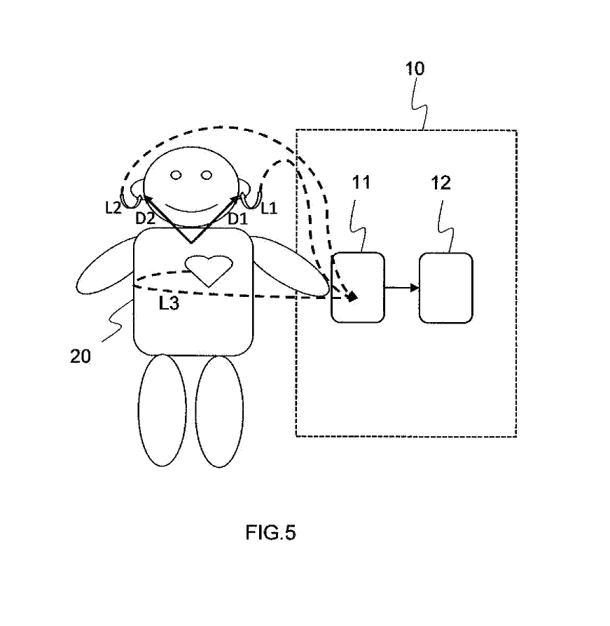

FIG. 5 is a schematic diagram of an apparatus for measuring a physiological signal according to one embodiment. In this embodiment, the apparatus for measuring a physiological signal 10 includes a measuring unit 11 and a signal-analyzing unit 12. The measuring unit 11 has a first signal-measuring end L1 and a second signal-measuring end L2. The first signal-measuring end L1 and the second signal-measuring end L2 contact ears (two symmetrical portions) of a tested body (living being 20) to obtain a first pulse signal and a second pulse signal of the ears (two symmetrical portions), respectively. The signal-analyzing unit 12 is coupled to the measuring unit 11. The signal-analyzing unit 12 obtains a first crest-to-cycle ratio and a second crest-to-cycle ratio (first physiological data group) based on the first pulse signal and the second pulse signal further to determine the degree of arteriosclerosis (physiological condition) of the tested body (living being 20) according to the first crest-to-cycle ratio and the second crest-to-cycle ratio (first physiological data group). The first signal-measuring end L1 and the second signal-measuring end L2 include a photoplethysmography for obtaining the first pulse signal and the second pulse signal of the ears (two symmetrical portions), respectively, using infrared light of about 940 nm wavelengths.

The measuring unit 11 further includes a third signal-measuring end L3 for measuring an ECG signal of the tested body (living being 20). The signal-analyzing unit 12 obtains a first pulse wave velocity, a second pulse wave velocity, a first multiscale entropy coefficient, a second multiscale entropy coefficient, a first pearson correlation coefficient, and a second pearson correlation coefficient (second physiological data group) based on the first pulse signal and the second pulse signal along with the ECG signal further to determine the degree of arteriosclerosis (physiological condition) of the tested body (living being 20) according to the first pulse wave velocity, the second pulse wave velocity, the first multiscale entropy coefficient, the second multiscale entropy coefficient, the first pearson correlation coefficient, and the second pearson correlation coefficient (second physiological data group).

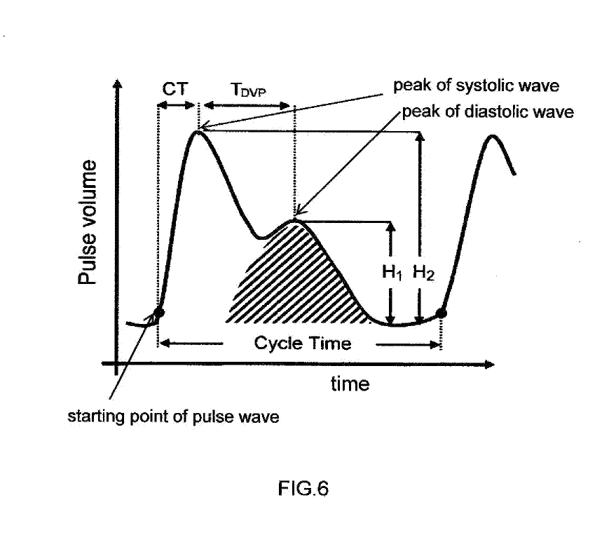

FIG. 6 is a schematic diagram of calculation of a first crest-to-cycle ratio of a first pulse signal or a second crest-to-cycle ratio of a second pulse signal according to one embodiment. In this embodiment, the apparatus for measuring a physiological signal 10 includes a measuring unit 11 and a signal-analyzing unit 12. The measuring unit 11 has a first signal-measuring end L1 and a second signal-measuring end L2. The first signal-measuring end L1 and the second signal-measuring end L2 contact two symmetrical portions of a living being 20 to obtain a first pulse signal and a second pulse signal of the two symmetrical portions, respectively. The signal-analyzing unit 12 is coupled to the measuring unit 11. The signal-analyzing unit 12 obtains a first crest-to-cycle ratio and a second crest-to-cycle ratio based on the first pulse signal and the second pulse signal further to determine a physiological condition of the living being 20 according to the first crest-to-cycle ratio and the second crest-to-cycle ratio.

First, the first signal-measuring end L1 and the second signal-measuring end L2 contact ears (two symmetrical portions) of a tested body (living being 20), respectively. In a certain time (such as 5 minutes), the first pulse signal and the second pulse signal of the ears (two symmetrical portions) are measured. Since the waveform of the first pulse signal of the ears (two symmetrical portions) is similar to that of the second pulse signal, the first pulse signal or the second pulse signal of the ears (two symmetrical portions) is taken for example.

Second, the signal-analyzing unit 12 obtains a crest time (CT) measured from a starting point of a pulse wave to a peak of a systolic wave and a cycle time based on the first pulse signal or the second pulse signal of the ears (two symmetrical portions), respectively. Afterwards, the first crest-to-cycle ratio (CTR₁) and the second crest-to-cycle ratio (CTR₂) are obtained by dividing the crest time by the cycle time, respectively, as shown in the formula (4) and the formula (5).

$$\begin{matrix}{CTR}_{1} = \frac{CT 1}{Cycle Time 1} \times 100 \% & ( 4 ) \\ {CTR}_{2} = \frac{CT 2}{Cycle Time 2} \times 100 \% & ( 5 )\end{matrix}$$

If the first crest-to-cycle ratio or the second crest-to-cycle ratio of the tested body (living being 20) exceeds the crest-to-cycle ratio in a normal state, the tested body (living being 20) has suffered from arteriosclerosis (physiological condition). Further, no matter whether the first crest-to-cycle ratio or the second crest-to-cycle ratio of the tested body (living being 20) exceeds the crest-to-cycle ratio in the normal state, if the difference between the first crest-to-cycle ratio and the second crest-to-cycle ratio of the tested body (living being 20) is too great, the tested body (living being 20) has suffered from arteriosclerosis (physiological condition) as well.

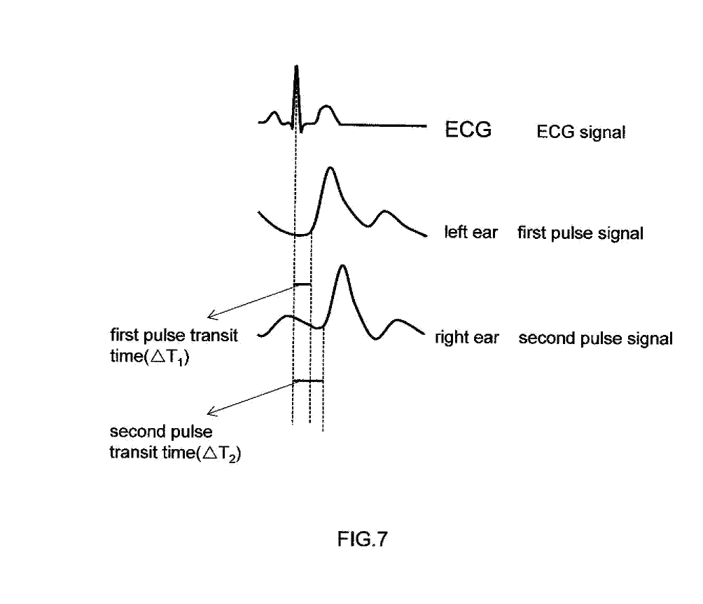

FIG. 7 is a schematic diagram of calculation of a first pulse wave velocity based on a first pulse signal along with an ECG signal and a second pulse wave velocity based on a second pulse signal along with an ECG signal according to one embodiment. In this embodiment, the apparatus for measuring a physiological signal 10 includes a measuring unit 11 and a signal-analyzing unit 12. The measuring unit 11 has a first signal-measuring end L1, a second signal-measuring end L2, and a third signal-measuring end L3. The first signal-measuring end L1 and the second signal-measuring end L2 contact two symmetrical portions of a living being 20 to obtain a first pulse signal and a second pulse signal of the two symmetrical portions, respectively. The third signal-measuring end L3 is used for measuring an ECG signal of the living being 20. The signal-analyzing unit 12 is coupled to the measuring unit 11. The signal-analyzing unit 12 obtains a first pulse wave velocity and a second pulse wave velocity based on the first pulse signal and the second pulse signal along with the ECG signal, respectively, further to determine the physiological condition of the living being 20 according to the first pulse wave velocity and the second pulse wave velocity.

First, a first distance D1 from the suprasternal notch to one ear and a second distance D2 from the suprasternal notch to the other ear of the tested body (living being 20) are measured, respectively, using a tape (please refer to FIG. 5), and then the first distance D1 and the second distance D2 are input into the apparatus for measuring a physiological signal 10.

Second, the first signal-measuring end L1 and the second signal-measuring end L2 contact the ears (two symmetrical portions) of the tested body (living being 20). In a certain time (such as 5 minutes), the first pulse signal and the second pulse signal of the ears (two symmetrical portions) are measured. The third signal-measuring end L3 is used for measuring the ECG signal of the tested body (living being 20).

Third, the signal-analyzing unit 12 obtains a first pulse transit time (ΔT₁) between the peak of R-wave of the ECG signal and the starting point of the first pulse signal based on the first pulse signal of the ears (two symmetrical portions) along with the ECG signal, and obtains a second pulse transit time (ΔT₂) between the peak of R-wave of the ECG signal and the starting point of the second pulse signal based on the second pulse signal along with the ECG signal. Afterwards, a first pulse wave velocity (PWV₁) is obtained by dividing the first distance D1 by the first pulse transit time (ΔT₁) as shown in the formula (6), and a second pulse wave velocity (PWV₂) is obtained by dividing the second distance D2 by the second pulse transit time (ΔT₂) as shown in the formula (7).

$$\begin{matrix}{PWV}_{1} = \frac{{D}_{1}}{Δ {T}_{1}} & ( 6 ) \\ {PWV}_{2} = \frac{{D}_{2}}{Δ {T}_{2}} & ( 7 )\end{matrix}$$

If the first pulse wave velocity or the second pulse wave velocity of the tested body (living being 20) exceeds the pulse wave velocity in a normal state (in an ideal state, the pulse wave velocity of the ear based on the ECG signal is 1.20 m/sec; the pulse wave velocity of the finger based on the ECG signal is 4.48 m/sec; the pulse wave velocity of the toe based on the ECG signal is 4.84 m/sec), the tested body (living being 20) has suffered from arteriosclerosis (physiological condition). Further, no matter whether the first pulse wave velocity or the second pulse wave velocity of the tested body (living being 20) exceeds the pulse wave velocity in the normal state, if the difference between the first pulse wave velocity and the second pulse wave velocity of the tested body (living being 20) is too great, the tested body (living being 20) has suffered from arteriosclerosis (physiological condition) as well.

According to the embodiments, the first crest-to-cycle ratio, the second crest-to-cycle ratio, the first pulse wave velocity, and the second pulse wave velocity of the two symmetrical portions (such as ears) of the tested body (living being 20) can be measured in a certain time (such as 5 minutes) further to determine the degree of arteriosclerosis (physiological condition) of the tested body (living being 20) according to one or all of the first crest-to-cycle ratio, the second crest-to-cycle ratio, the first pulse wave velocity, and the second pulse wave velocity.

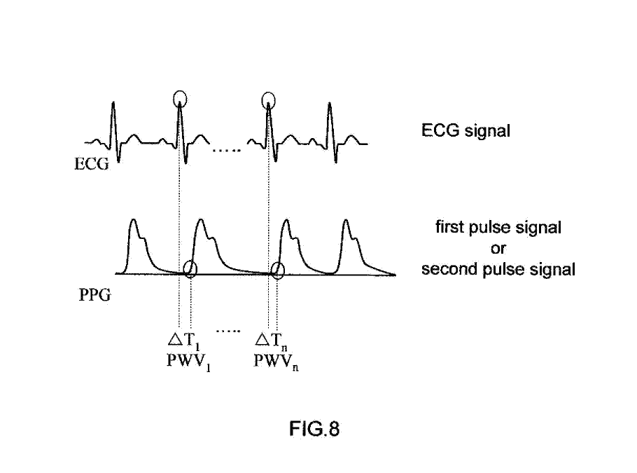

FIG. 8 is a schematic diagram of calculation of a first multiscale entropy coefficient based on a first pulse signal along with an ECG signal or a second multiscale entropy coefficient based on a second pulse signal along with an ECG signal according to one embodiment. In this embodiment, the apparatus for measuring a physiological signal 10 includes a measuring unit 11 and a signal-analyzing unit 12. The measuring unit 11 has a first signal-measuring end L1, a second signal-measuring end L2, and a third signal-measuring end L3. The first signal-measuring end L1 and the second signal-measuring end L2 contact two symmetrical portions of a living being 20 to obtain a first pulse signal and a second pulse signal of the two symmetrical portions, respectively. The third signal-measuring end L3 is used for measuring an ECG signal of the living being 20. The signal-analyzing unit 12 is coupled to the measuring unit 11. The signal-analyzing unit 12 obtains a first pulse wave velocity and a second pulse wave velocity based on the first pulse signal and the second pulse signal along with the ECG signal, respectively. Further, the signal-analyzing unit 12 obtains a first multiscale entropy coefficient and a second multiscale entropy coefficient based on the first pulse wave velocity and the second pulse wave velocity, respectively, using empirical mode decomposition (EMD) and a multiscale entropy analysis (complexity analysis), thus to determine the physiological condition of the living being 20. Since the waveform of the first pulse signal of the ears (two symmetrical portions) is similar to that of the second pulse signal, the first pulse signal along with the ECG signal or the second pulse signal along with the ECG signal of the ears (two symmetrical portions) is taken for example.

First, the signal-analyzing unit 12 gathers successive pulse wave velocities of the ears (two symmetrical portions) in a certain time (such as 5 minutes) to make up a series A {PWV₁, PWV₂, . . . , PWVₙ}. Since unsteady-state characteristic of physiological signals may increase the degree of irregularity of the time series thus to affect accuracy of the multiscale entropy analysis (complexity analysis) for different scales, trend is removed from the series A{PWV₁, PWV₂, . . . , PWVₙ} to obtain a series B {X₁,X₂, . . . , Xₙ} using the empirical mode decomposition (EMD) before the operation of the multiscale entropy analysis (complexity analysis) so as to obtain an accurate result after the operation of the multiscale entropy analysis (complexity analysis).

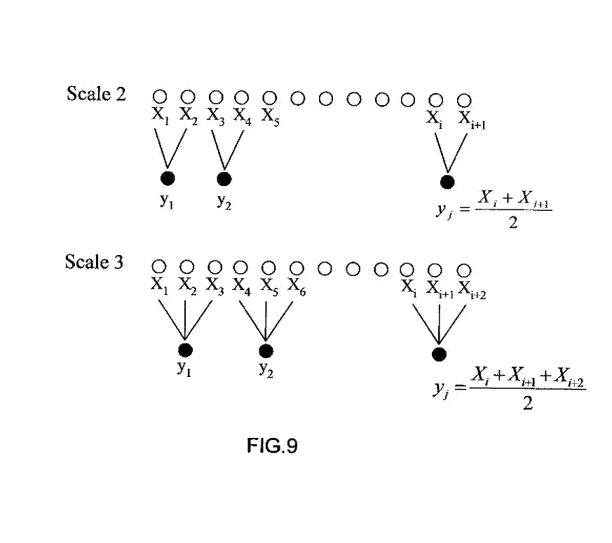

FIG. 9 is a schematic diagram of calculation of a coarse-grained technology according to one embodiment. First, in this embodiment, a series B {X₁,X₂, . . . , Xₙ} is transformed into signals in different scales (such as scale 2, scale 3 and so on) using a coarse-grained technology to show difference. Second, the time series in different scales {yⱼ^{(τ)}} after the coarse-grained operation are analyzed using sample entropy (SE). The sample entropies in different scales are multiscale entropy coefficients of the series B {X₁,X₂, . . . , Xₙ} where τ is a scale factor 1, 2, 3 and so on. If τ=2, yⱼ=Xᵢ+Xᵢ₊₁/2; if τ=3, yⱼ=Xᵢ+Xᵢ₊₁+Xᵢ₊₂/2; the rest may be inferred. The details are shown as the formula (8) and the formula (9).

$$\begin{matrix}{y}_{j}^{( 2 )} = \frac{{X}_{i} + {X}_{i + 1}}{2} & ( 8 ) \\ {y}_{j}^{( 3 )} = \frac{{X}_{i} + {X}_{i + 1} + {X}_{i + 2}}{2} & ( 9 )\end{matrix}$$

Third, the series B {X₁,X₂, . . . , Xₙ} in different scales is decomposed into samples consisting of m points by a length N, and thus N−m+1 different samples can be obtained. A sample space X can be obtained using the following formula (10).

$$X = [ \begin{matrix}{X}_{1} & {X}_{2} & … & {X}_{m} \\ {X}_{2} & {X}_{3} & … & {X}_{m + 1} \\ {X}_{N - m + 1} & {X}_{N - m + 2} & & {X}_{N}\end{matrix} ] \qquad ( 10 )$$

Fourth, the series B {X₁,X₂, . . . , Xₙ} is analyzed using sample entropy including the following steps (a)-(f). However, the sequence of the steps is not limited.
(a) obtaining distances between the samples, which can be shown as Dij=|Xi-Xj| where i≠j;
(b) transforming the distance into similarity between the samples using the formula Dij(r)=G(Dij), where G(Dij) is a Heaviside function defined as the formula (11);

$$G ( Dij ) = \{ \begin{matrix}1 , {D}_{ij} < r \\ 0 , {D}_{ij} > r\end{matrix} \qquad ( 11 )$$

(c) obtaining an average value Cₘ(r) using the formula (12);

$${C}_{m} ( r ) = \frac{1}{N - m + 1} {\sum }_{i = 1}^{N - m + 1} {D}_{ij} \qquad ( 12 )$$

(d) obtaining an average similarity Cₘ₋₁(r) between the samples with a length m using the formula (13);

$${C}_{m + 1} ( r ) = \frac{1}{N - m} {\sum }_{i = 1}^{N - m} {C}_{m} ( r ) \qquad ( 13 )$$

(e) repeating the steps (a)˜(d) to calculate Cₘ₊₁(r) with a length m+1;
(f) obtaining the sample entropy (SE) S_{E}(m,r) based on Cₘ(r) and Cₘ₊₁(r) using the formula (14);

$${S}_{E} ( m , r ) = - log \frac{{C}_{m + 1} ( r )}{{C}_{m} ( r )} \qquad ( 14 )$$

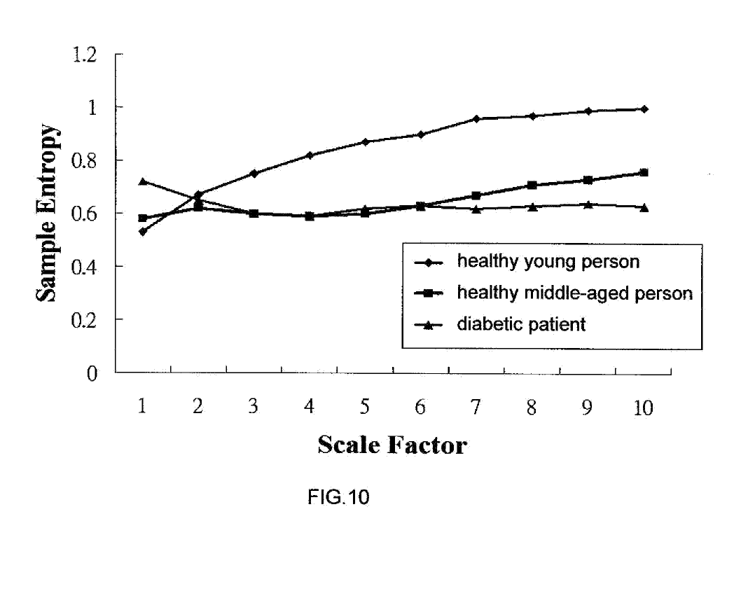

FIG. 10 is a schematic diagram of sample entropy related to scale variability according to one embodiment. In this embodiment, the sample entropies of the series B {X₁,X₂, . . . , Xₙ} in different scales are obtained according to the steps (a)˜(f) (using the formulas (8)˜(14)), and thus the curved line showing the sample entropy related to the scale variability can be obtained, i.e. the multiscale entropy analysis (complexity analysis).

According to the schematic diagram of the multiscale entropies for a healthy young person, a healthy middle-aged person, and a diabetic patient (please refer to FIG. 10), it can be concluded that the healthy young person has the most complex artery-blood-vessel function and the healthy middle-aged person and the diabetic patient follow successively. From FIG. 10 it can be seen that the complexity of the artery-blood-vessel function for the diabetic patient is relatively low, and therefore diabete mellitus is an important risk factor of the arteriosclerosis.

In addition, many patients (such as the diabetic patients) may easily suffer from autonomic instability as well as the arteriosclerosis. The embodiments of the invention may quantify the couping degree between the heart rate and the vessel thus to indicate the physical condition of the person. Since the waveform of the first pulse signal of the ears (two symmetrical portions) is similar to that of the second pulse signal, the first pulse signal along with the ECG signal or the second pulse signal along with the ECG signal of the ears (two symmetrical portions) is taken for example.

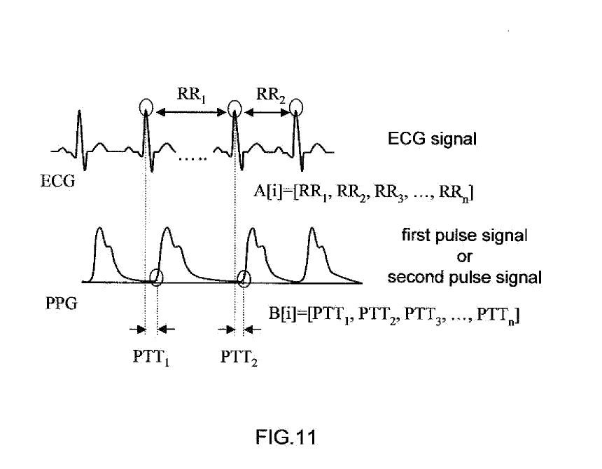

FIG. 11 is a schematic diagram of calculation of a first pearson correlation coefficient based on a first pulse signal along with an ECG signal or a second pearson correlation coefficient based on a second pulse signal along with an ECG signal according to one embodiment.

First, the signal-analyzing unit 12 gathers successive RR-interval (RRI) signals of the ears (two symmetrical portions) in a certain time (such as 5 minutes) to make up a series A[i] {RR₁,RR₂,RR₃, . . . RRₙ}, and gathers successive pulse transit time (PTT) of the first pulse signal or the second pulse signal of the ears (two symmetrical portions) in a certain time (such as 5 minutes) to make up a series B[i] {PTT₁, PTT₂, PTT₃, . . . , PTTₙ}, where the series A[i] {RR₁,RR₂,RR₃, . . . RRₙ} indicates the heart rate variability and the series B[i] {PTT₁, PTT₂, PTT₃, . . . , PTTₙ} indicates the artery-blood-vessel variability. Second, the signal-analyzing unit 12 obtains the first pearson correlation coefficient and the second pearson correlation coefficient based on the series A[i] {RR₁,RR₂,RR₃, . . . RRₙ} and the series B[i] {PTT₁, PTT₂, PTT₃, . . . , PTTₙ} of the ears, respectively, to help analyzing the couping degree between the heart rate and the artery-blood-vessel which is defined as correlation of PTT and RRI (CPR) obtained using the formula (15).

$$CPR ≅ \frac{{\sum }_{i = 1}^{1000} [ A ( i ) - mean ( A ( i ) ) ]}{\sqrt{{\sum }_{i = 1}^{1000} {[ A ( i ) - mean ( A ( i ) ) ]}^{2}} \sqrt{{\sum }_{i = 1}^{1000} 1 [ B ( i ) - {mean ( ( B ( i ) ) ]}^{2}}} \qquad ( 15 )$$

According to the analysis of the pearson correlation coefficients for the healthy young person, the healthy middle-aged person, and the diabetic patient, it can be concluded that, the pearson correlation coefficient of the healthy young person is 0.15, presenting a high positive correlation; the pearson correlation coefficient of the healthy middle-aged person is 0.00, presenting a negative correlation; the pearson correlation coefficient of the diabetic patient is −0.15, presenting a high negative correlation. Accordingly, age growth and diabete mellitus may indeed affect the couping between the heart rate and the artery-blood-vessel, and the pearson correlation coefficient is low as well.

Further, the invention provides a method for measuring a physiological signal including the following step: contacting at least two symmetrical portions of a living being to obtain a first pulse signal and a second pulse signal of the two symmetrical portions, respectively; obtaining a physiological data based on the first pulse signal and the second pulse signal, respectively, further to determine a physiological condition of the living being according to the physiological data. Please refer to FIG. 5 through FIG. 11. Steps (a)˜(k) may be described below while the sequence of the steps is not limited.
(a): as shown in FIG. 5, measuring a first distance D1 and a second distance D2 from the suprasternal notch to the ears of the tested body (living being 20), respectively, using a tape, and inputting the first distance D1 and the second distance D2 into the apparatus for measuring a physiological signal 10;
(b): contacting the ears (two symmetrical portions) of the tested body (living being 20) in a certain time (such as 5 minutes) further to obtain a first pulse signal and a second pulse signal of the ears (two symmetrical portions), respectively;
(c): measuring an ECG signal of the tested body (living being 20);
(d): as shown in FIG. 6, obtaining a first crest-to-cycle ratio and a second crest-to-cycle ratio (first physiological data group) based on the first pulse signal and the second pulse signal;
(e): as shown in FIG. 7, obtaining a first pulse transit time between the peak of R-wave of the ECG signal and the starting point of the first pulse signal based on the first pulse signal along with the ECG signal and obtaining a second pulse transit time between the peak of R-wave of the ECG signal and the starting point of the second pulse signal based on the second pulse signal along with the ECG signal; obtaining a first pulse wave velocity by dividing the first distance D1 by the first pulse transit time as shown in the formula (6) and obtaining a second pulse wave velocity by dividing the second distance D2 by the second pulse transit time as shown in the formula (7);
(f): as shown in FIG. 8, gathering successive pulse wave velocities of the ears (two symmetrical portions) in a certain time (such as 5 minutes) to make up a series A {PWV₁, PWV₂, . . . , PWVₙ};
(g): removing trend from the series A {PWV₁, PWV₂, . . . , PWVₙ} to obtain a series B{X₁,X₂, . . . , Xₙ} using empirical mode decomposition (EMD);
(h): as shown in FIG. 9, transforming the series B {X₁,X₂, . . . , Xₙ} into signals in different scales (such as scale 2, scale 3 and so on) using a coarse-grained technology to show difference;
(i): as shown in FIG. 10, analyzing the time series in different scales {yⱼ^{(τ)}} after the coarse-grained operation using sample entropy (SE) thus to obtain the curved line showing the sample entropy SE related to the scale T variability;
(j): as shown in FIG. 11, gathering successive RR-interval (RRI) signals of the ears (two symmetrical portions) in a certain time (such as 5 minutes) to make up a series A[i] {RR₁,RR₂,RR₃, . . . RRₙ} and gathering successive pulse transit time of the first pulse signal or the second pulse signal of the ears (two symmetrical portions) in a certain time (such as 5 minutes) to make up a series B[i] {PTT₁, PTT₂, PTT₃, . . . , PTTₙ}; obtaining a first pearson correlation coefficient and a second pearson correlation coefficient based on the series A[i] {RR₁,RR₂,RR₃, . . . RRₙ} and the series B[i] {PTT₁, PTT₂, PTT₃, . . . , PTTₙ} of the ears, respectively, to help analyzing the couping degree between the heart rate and the artery-blood-vessel which is defined as correlation of PTT and RRI (CPR) obtained using the formula (15);
(k): determining the degree of arteriosclerosis (physiological condition) of the tested body (living being 20) according to the first crest-to-cycle ratio, the second crest-to-cycle ratio, the first pulse wave velocity, the second pulse wave velocity, the first multiscale entropy coefficient, the second multiscale entropy coefficient, the first pearson correlation coefficient, and the second pearson correlation coefficient.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. An apparatus for measuring a physiological signal, comprising:
a measuring unit having at least one first signal-measuring end and at least one second signal-measuring end, the first signal-measuring end and the second signal-measuring end contacting at least two symmetrical portions of a living being to obtain at least one first pulse signal and at least one second pulse signal of the two symmetrical portions, respectively; and
a signal-analyzing unit coupled to the measuring unit, the signal-analyzing unit obtaining at least one physiological data based on the first pulse signal and the second pulse signal, respectively, further to determine a physiological condition of the living being according to the physiological data.

2. The apparatus for measuring a physiological signal according to claim 1, wherein the physiological data comprises at least one first physiological data group and at least one second physiological data group.

3. The apparatus for measuring a physiological signal according to claim 2, wherein the first physiological data group comprises at least one first crest-to-cycle ratio and at least one second crest-to-cycle ratio.

4. The apparatus for measuring a physiological signal according to claim 2, wherein the measuring unit further comprises a third signal-measuring end for measuring an ECG signal of the living being.

5. The apparatus for measuring a physiological signal according to claim 4, wherein the signal-analyzing unit obtains the second physiological data group based on the first pulse signal and the second pulse signal along with the ECG signal further to determine the physiological condition of the living being according to the second physiological data group.

6. The apparatus for measuring a physiological signal according to claim 2, wherein the second physiological data group comprises at least one first pulse wave velocity, at least one second pulse wave velocity, at least one first complexity coefficient, at least one second complexity coefficient, at least one first pearson correlation coefficient, and at least one second pearson correlation coefficient.

7. The apparatus for measuring a physiological signal according to claim 6, wherein the signal-analyzing unit obtains at least one pulse transit time and the first pearson correlation coefficient based on the first pulse signal along with the ECG signal further to obtain the first pulse wave velocity based on the first pulse transit time.

8. The apparatus for measuring a physiological signal according to claim 6, wherein the signal-analyzing unit obtains at least one second pulse transit time and the second pearson correlation coefficient based on the second pulse signal along with the ECG signal further to obtain the second pulse wave velocity based on the second pulse transit time.

9. The apparatus for measuring a physiological signal according to claim 6, wherein the signal-analyzing unit obtains the first complexity coefficient and the second complexity coefficient based on the first pulse wave velocity and the second pulse wave velocity, respectively, using empirical mode decomposition and a complexity analysis.

10. The apparatus for measuring a physiological signal according to claim 6, wherein the first complexity coefficient and the second complexity coefficient comprises at least one first multiscale entropy coefficient and at least one second multiscale entropy coefficient, respectively.

11. A method for measuring a physiological signal, comprising the steps of:
contacting at least two symmetrical portions of a living being to obtain at least one first pulse signal and at least one second pulse signal of the two symmetrical portions, respectively; and
obtaining at least one physiological data based on the first pulse signal and the second pulse signal, respectively, further to determine a physiological condition of the living being according to the physiological data.

12. The method for measuring a physiological signal according to claim 11, wherein the physiological data comprises at least one first physiological data group and at least one second physiological data group.

13. The method for measuring a physiological signal according to claim 12, wherein the first physiological data group comprises at least one first crest-to-cycle ratio and at least one second crest-to-cycle ratio.

14. The method for measuring a physiological signal according to claim 12, further comprising the step of measuring an ECG signal of the living being.

15. The method for measuring a physiological signal according to claim 14, further comprising the step of obtaining the second physiological data group based on the first pulse signal and the second pulse signal along with the ECG signal further to determine the physiological condition of the living being according to the second physiological data group.

16. The method for measuring a physiological signal according to claim 12, wherein the second physiological data group comprises at least one first pulse wave velocity, at least one second pulse wave velocity, at least one first complexity coefficient, at least one second complexity coefficient, at least one first pearson correlation coefficient, and at least one second pearson correlation coefficient.

17. The method for measuring a physiological signal according to claim 16, further comprising the step of obtaining at least one first pulse transit time and the first pearson correlation coefficient based on the first pulse signal along with the ECG signal further to obtain the first pulse wave velocity based on the first pulse transit time.

18. The method for measuring a physiological signal according to claim 16, further comprising the step of obtaining at least one second pulse transit time and the second pearson correlation coefficient based on the second pulse signal along with the ECG signal further to obtain the second pulse wave velocity based on the second pulse transit time.

19. The method for measuring a physiological signal according to claim 16, further comprising the step of obtaining the first complexity coefficient and the second complexity coefficient based on the first pulse wave velocity and the second pulse wave velocity, respectively, using empirical mode decomposition and a complexity analysis.

20. The method for measuring a physiological signal according to claim 16, wherein the first complexity coefficient and the second complexity coefficient comprises at least one first multiscale entropy coefficient and at least one second multiscale entropy coefficient, respectively.
